(19) United States
(12) Patent Application Publication
(10) Pub. No.: US 2024/0122982 A1
(43) Pub. Date: Apr. 18, 2024

(54) CHIMERIC ANTIGEN RECEPTOR FUSION PROTEIN CO-EXPRESSING IL-7 AND CCR2B, AND APPLICATION THEREOF

(71) Applicant: GUANGZHOU BIO-GENE TECHNOLOGY CO., LTD, Guangzhou (CN)

(72) Inventors: Guangchao LI, Guangzhou (CN); Min LUO, Guangzhou (CN); Wen DING, Guangzhou (CN); Xuejun WANG, Guangzhou (CN)

(21) Appl. No.: 18/278,163

(22) PCT Filed: Mar. 30, 2021
(86) PCT No.: PCT/CN2021/083847

§ 371 Date: Aug. 21, 2023

(30) Foreign Application Priority Data
Feb. 22, 2021 (CN) 202110196691.7

(51) Int. Cl. A61K 35/17; A61K 39/00; A61P 35/00; C07K 14/54; C07K 14/705; C07K 14/715; C07K 16/30

(52) U.S. Cl. CPC A61K 35/17; A61K 39/4611; A61K 39/4631; A61P 35/00; C07K 14/5418; C07K 14/70517; C07K 14/7158; C07K 16/3084; A61K 2039/5156; A61K 2239/15; C07K 2317/622; C07K 2319/02; C07K 2319/03

(57) ABSTRACT

A chimeric antigen receptor fusion protein co-expressing IL-7 and CCR2b, and application thereof are provided. The fusion protein includes a chimeric antigen receptor, a 2A peptide, IL-7, a 2A peptide and CCR2b which are sequentially linked in series.

### CROSS-REFERENCE TO RELATED APPLICATIONS

This application is a U.S. national phase application under 35 U.S.C. § 371 which claims priority to International Patent Application No. PCT/CN2021/083847, filed on Mar. 30, 2021, which claims the priority benefit of Chinese Patent Application No. 202110196691.7, filed on Feb. 22, 2021. The contents of the above identified applications are hereby incorporated herein in their entireties by reference.

### SEQUENCE LISTING

This application contains a Sequence Listing in computer readable form submitted via EFS-Web. The entire contents of the ASCII text file entitled “ACL0142US Sequence Listing.txt” created on Aug. 4, 2023, having a size of 29,655 bytes, is incorporated herein by reference.

### TECHNICAL FIELD

The present disclosure relates to the field of biomedicine, and specifically to a chimeric antigen receptor fusion protein co-expressing IL-7 and CCR2b, and application thereof.

### BACKGROUND

Recently, a biological therapy for a tumor has become a new therapeutic method followed surgery therapy and chemoradiotherapy. The biological therapy is a kind of therapy that uses biological methods to adjust the “anti-cancer mechanism” of an organism, thereby making it balanced and stable. The biological therapy includes a cell therapy, a gene therapy, an antibody therapy and a cytokine therapy.

Among them, a chimeric antigen receptor T-cell (CAR-T) therapy is a promising one. In the basic design, a chimeric antigen receptor (CAR) includes a tumor associated antigen-binding region that is usually derived from the single-chain variable fragment (scFv) segment of an antigen-binding region of an antibody, an extracellular hinge region, a transmembrane region, and an intracellular signaling region. Upon such a receptor is expressed by T cells, a single fusion molecule specifically binds to an antigen and activates T cells, so that the T cell modified by the chimeric antigen receptor has the specificity of an antibody and the cytotoxic effect of effector T cells. CAR, once bound to tumor-associated antigen (TAA), can activate T cells through CD3 or an intracellular region of high-affinity receptor FceR1, exhibiting CAR-dependent cell killing, proliferation and cytokine release. Meanwhile, CAR-T cells can be expanded by more than 1000 times. It has been found in clinical trials that high expression level of CAR can still be detectable in patients in six months after inputting CAR-T cells. In short, CAR-T cells have targeting ability, killing activity and persistence superior to those of conventionally used immune cells.

CAR-T cells have remarkable therapeutic effect on cancer immunotherapy, especially on the treatment of leukemia. However, there are two major problems that need to be solved in the treatment of a solid tumor by CAR-T, including survival of CAR-T cells in a solid tumor site and effective migration of CAR-T cells to a solid tumor site.

### SUMMARY

In a first aspect, the present disclosure relates to a fusion protein including a chimeric antigen receptor The fusion protein includes a chimeric antigen receptor, a 2A peptide, IL-7, a 2A peptide and CCR2b which are sequentially linked in series.

In a second aspect, the present disclosure relates to an isolated nucleic acid, expressing the fusion protein as described above.

In a third aspect, the present disclosure relates to a vector, including the nucleic acid as described above.

In a fourth aspect, the present disclosure relates to a T cell, including the nucleic acid as described above or the vector as described above.

In a fifth aspect, the present disclosure relates to a composition, including the T cell as described above and a pharmaceutically acceptable carrier.

In a sixth aspect, the present disclosure relates to use of the T cell as described above or the composition as described above in the preparation of a medicament for preventing and/or treating a solid tumor.

### BRIEF DESCRIPTION OF THE DRAWINGS

In order to illustrate the embodiments of the present disclosure or the technical solutions of the prior art more clearly, the drawings that need to be used in the description of the specific embodiments or the prior art will be described briefly below. Apparently, the following described drawings are merely for some embodiments of the present disclosure, and other drawings can also be derived by those of ordinary skill in the art based on these drawings without any creative effort.

FIG. 1 shows a structural representation comparison between a conventional CAR and a CAR co-expressing IL-7 and CCR2b (7×2b) in an embodiment of the present disclosure.

FIG. 2A shows the structure of a conventional CAR targeting GD2 in an embodiment of the present disclosure.

FIG. 2B shows the structure of a CAR co-expressing IL-7 and CCR2b (7×2b) in an embodiment of the present disclosure.

FIG. 3 shows the expression of CAR-T detected by flow cytometry and the secretion of IL-7 detected by ELISA in an embodiment of the present disclosure.

FIG. 4 shows the expression of GD2 antigen in melanoma and neuroblastoma detected by flow cytometry in an embodiment of the present disclosure.

FIG. 5 shows the secretion levels of IFN-γ by the CAR-T cell after co-incubation with tumor cells detected by ELISA in an embodiment of the present disclosure.

FIG. 6 shows the killing efficiency of the CAR-T cell detected by luciferase assay in an embodiment of the present disclosure.

FIG. 7 shows the secretion levels of CCL2 by tumor cells detected by ELISA in an embodiment of the present disclosure.

FIG. 8 shows the effect of the CAR-T cell on promoting proliferation and migration in an embodiment of the present disclosure, in which (A) shows evaluation of the expansion efficiency of the CAR-T cell; (B) shows effect of the CAR-T cell supernatant on stimulating T cell expansion; (C) shows analysis of Tscm subset; and (D) shows evaluation of the chemotactic ability of the tumor cell supernatant to the CAR-T cell.

FIG. 9 shows secretion level of CLL2 by the IMR-32-CCL2 cell stably transfected with CLL2 gene detected by ELISA in an embodiment of the present disclosure.

FIG. 10 shows an in vivo imaging of the mice and analysis data of the bioluminescence imaging results.

FIG. 11 shows secretion levels of cytokines by mice after being treated with CAR-T in an embodiment of the present disclosure.

FIG. 12 shows expression of human CD3 in the spleen and tumor mass of mice detected by immunohistochemical tests in an embodiment of the present disclosure.

### DETAILED DESCRIPTION

Reference to the embodiments of the present disclosure will now be provided in detail, in which one or more examples are described below. Each example is provided for explaining but not intended to limit the present disclosure. Indeed, it would be apparent to those skilled in the art that various modifications and variations can be made to the present disclosure without departing from the scope or spirit of the present disclosure. For example, a feature stated or described as a part of one embodiment may be used in another embodiment to form a further embodiment.

Unless otherwise defined, all technical and scientific terms used herein have the same meaning as normally understood by those skilled in the art to which the present disclosure belongs. The terms used herein in the specification of the present disclosure are for the purpose of describing specific embodiments only and are not intended to limit the present disclosure. The term “and/or” as used herein includes any and all combinations of one or more related listed items.

The present disclosure relates to a fusion protein including a chimeric antigen receptor. The fusion protein includes a chimeric antigen receptor, a 2A peptide, IL-7, a 2A peptide and CCR2b which are sequentially linked in series.

Interleukin-7 (IL7) is a kind of multi-effect cytokines with broad immune effects, which can affect the growth, survival and differentiation of B cells and T cells, and also have direct or indirect effects on anti-tumor.

Chemokine receptor type 2 (CCR2) is a receptor of monocyte chemoattractant protein-1 (MCP-1). It has been confirmed that CCR2 and its ligand MCP-1 can play an important role in the pathology of inflammatory diseases, such as resistance to Mycobacterium tuberculosis during lung transplantation, lipopolysaccharide-induced death, delayed atopic dermatitis, etc.

In order to improve the therapeutic effect of CAR-T cell on a solid tumor, a CAR-T cell co-expressing IL-7 and CCR2b (7×2b CAR-T) is constructed and prepared in the present disclosure. The 7×2b CAR-T cell can have viability, chemotactic ability and subset distribution superior to those of the conventional CART cell, so that it is expected to achieve superior anti-tumor effects in vivo, and provide a preclinical research basis for future clinical trials.

In some embodiments, the IL-7 has an amino acid sequence as set forth in SEQ ID NO: 2.

In some embodiments, the IL-7 further includes a signal peptide. The signal peptide optionally has an amino acid sequence as set forth in SEQ ID NO: 9.

In some embodiments, the CCR2b has an amino acid sequence as set forth in SEQ ID NO: 3.

In some embodiments, the 2A peptide is a T2A peptide having an amino acid sequence as set forth in SEQ ID NO: 4.

In some embodiments, the chimeric antigen receptor includes A) a sc-Fv region, B) a hinge region, C) a transmembrane region, and D) an intracellular signaling region.

In some embodiments, the hinge region is a hinge region of CD8α. In an embodiment, the hinge region has an amino acid sequence as set forth in SEQ ID NO: 5.

In some embodiments, the transmembrane region is any one selected from α, β or ζ chain of a T cell receptor, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2RO, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C.

In some embodiments, the transmembrane region is a CD8α transmembrane region having an amino acid sequence as set forth in SEQ ID NO: 6.

In some embodiments, the intracellular signaling region is any one selected from CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a CD83-specific binding ligand, CDS, ICAM-1, GITR, BAFFR, HVEM (LIGHTR), SLAMF7, NKp80 (KLRF1), CD160, CD19, CD4, CD8α, CD8β, IL2RO, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1(CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, PKCθ, FcεRIγ, ZAP70 and CD3ζ, or a combination thereof.

In some embodiments, the intracellular signaling region is 4-1BB and CD3ζ.

In some embodiments, the 4-1BB has an amino acid sequence as set forth in SEQ ID NO: 7.

In some embodiments, the CD3ζ has an amino acid sequence as set forth in SEQ ID NO: 8.

In some embodiments, the chimeric antigen receptor has a N-terminal signal peptide. The signal peptide optionally is a CD8α signal peptide. Further, the signal peptide has an amino acid sequence as set forth in SEQ ID NO: 10.

In some embodiments, the sc-Fv region is configured to target a surface marker on a solid tumor.

The sc-Fv region can be a chimeric, humanized, or human antibody fragment, and is an antigen-binding domain capable of recognizing a tumor. In some embodiments, the antigen-binding domain capable of recognizing a tumor can recognize any one of the following antigens: α-fetoprotein (AFP), α-actinin-4, A3, A33 antibody-specific antigen, ART-4, B7, Ba 733, BAGE, BrE3 antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon-specific antigen p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, a folate receptor, G250 antigen, GAGE, gp100, GRO-0, HLA-DR, HM1.24, human chorionic gonadotropin (HCG) and its subunits, HER2/neu, HMGB-1, hypoxic inducible factor-1 (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4 antigen, KS-1 antigen, KS1-4, Le-Y, LDR/FUT, macrophage migration inhibitory factor (MIF), MAGE, MAGE-3, MART1, MART-2, NY-ESO-1, TRAG-3, mCRP, MCP-1, MIP-1α, MIF, MUC1β, MUC2, MUC3, MUC4, MUC5ac, MUC13, MUC16, MUM-1/2, MUM-3, NCA66, NCA95, NCA90, pancreatic mucin, PD1 receptor, placental growth factor, p53, PLAGL2, prostatic acid phosphatase, PSA, PRAME, PSMA, PlGF, ILGF, ILGF-1R, IL-6, IL-25, RSS, RANTES, T101, SAGE, 5100, survivin, survivin-2B, TAC, TAG-72, cytotactin, TRAIL receptor, TNF-α, Tn antigen, Thomsen-Friedenreich antigen, tumor necrosis antigen, VEGFR, ED-B fibronectin, WT-1, 17-1A antigen, complement factors C3, C3a, C3b, C5a, C5, an angiogenesis marker, bc1-2, bcl-6, Kras, an oncogene marker, and an oncogene product.

In some embodiments, the sc-Fv region targets GD2. In an embodiment, the sc-Fv region has an amino acid sequence as set forth in SEQ ID NO: 1.

The present disclosure also relates to an isolated nucleic acid expressing the fusion protein as described above. The nucleic acid can be DNA or RNA.

The present disclosure further relates to a vector including the nucleic acid as described above.

In some specific embodiments of the present disclosure, the vector can be selected from a retrovirus vector, an adenovirus, an adenovirus-associated virus, or a CRISPR/CAS plasmid.

In some specific embodiments of the present disclosure, the retrovirus vector is a lentiviral vector.

In some specific embodiments of the present disclosure, the CRISPR/CAS plasmid can be any one selected from CRISPR/CAS-1, CRISPR/CAS-5, CRISPR/CAS-7, CRISPR/CAS-9, CRISPR/CAS-2, CRISPR/CAS-3 and CRISPR/CAS-10.

The present disclosure also relates to a T cell including the nucleic acid as described above or the vector as described above.

In some specific embodiments of the present disclosure, the T cell is any one of a helper T cell, a cytotoxic T cell, a memory T cell, a regulatory T cell, MAIT cell and γδT cell.

The present disclosure further relates to a composition including the T cell as described above and a pharmaceutically acceptable carrier.

The present disclosure further relates to use of the T cell as described above or the composition as described above in the preparation of a medicament for preventing and/or treating a solid tumor.

In the present disclosure, the solid tumor includes a tumor formed from the lesion of any one of bone, bone junction, muscle, lung, trachea, heart, spleen, arteries, veins, capillaries, lymph nodes, lymph vessels, lymph fluid, oral cavity, pharynx, esophagus, stomach, duodenum, small intestine, colon, rectum, anus, appendix, liver, bile, pancreas, parotid gland, sublingual gland, kidney, ureter, bladder, urethra, ovary, fallopian tube, uterus, vagina, genitalia, scrotum, testicle, vas deferens, penis, eyes, ears, nose, tongue, skin, brain, brainstem, medulla oblongata, spinal cord, cerebrospinal fluid, nerves, thyroid gland, parathyroid gland, adrenal gland, pituitary gland, pineal gland, pancreas islet, thymus, gonadal gland, sublingual gland, or parotid gland. In particular, the tumors intended to be targeted include, for example, cholangiocarcinoma, breast cancer, cervical cancer, chronic lymphocytic leukemia, chronic myeloid leukemia, colorectal cancer, endometrial cancer, esophageal cancer, stomach cancer, head and neck cancer, Hodgkin's lymphoma, lung cancer, medullary thyroid cancer, non-Hodgkin's lymphoma, multiple myeloma, kidney cancer, ovarian cancer, pancreatic cancer, glioma, melanoma, liver cancer, prostate cancer, or urinary bladder cancer.

In some embodiments, the solid tumor is a tumor with a high CCL2 level.

In some embodiments, the solid tumor is neuroblastoma or melanoma.

The present disclosure has the following beneficial advantages.

The conventional CAR-T cell cannot survive long enough in vivo and can be affected by various immunosuppressive factors in the tumor microenvironment, resulting in poor infiltration ability to the tumor and weak proliferation ability in the tumor microenvironment. A CAR-T cell co-expressing IL-7 and CCR2b, which is referred to as 7×2b CAR T cell, is constructed in the present disclosure. The 7×2b CAR T cell can be more effectively migrated to a solid tumor and can be efficiently proliferated in a tumor region.

Compared with the conventional CAR-T cell, the expression of CAR on the T cell surface and the specificity and effectiveness of CAR-T in killing tumor cells would not be affected by the secretion of IL-7 and the expression of CCR2b. Moreover, in the culture process without exogenous addition of IL-7, the 7×2b CAR T cell can exhibit a better proliferation ability, which would facilitate its survival in vivo and thus improve its clinical activity.

The embodiments of the present disclosure will be described in details in in combination with examples below.

### Example 1. Design of Chimeric Antigen Receptor

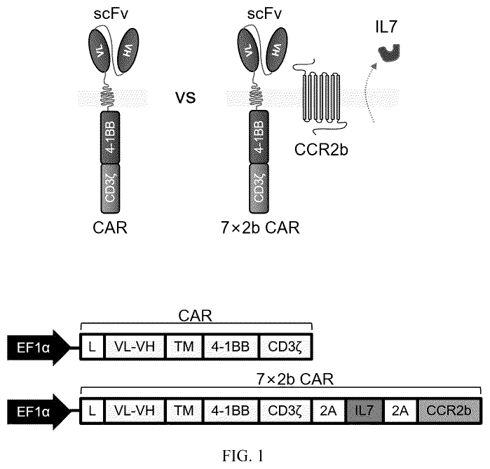

In this example, a GD2-targeting CAR was constructed by using the single chain antibody (scFv) of anti-GD2 antibody 14G2a as the antigen-binding domain, in combination with CD8α signal peptide, CD8α hinge region, CD8α transmembrane region, 4-1BB co-stimulatory domain and CD3 signaling domain, with a structural schematic diagram as shown in panel A of FIG. 1. In this example, a CAR co-expressing IL-7 and CCR2b (7×2b) was also constructed, with a structural schematic diagram as shown in panel B of FIG. 1.

The amino acid sequence of 14G2a-CAR is shown in SEQ ID NO: 11. The nucleotide sequence of 14G2a-CAR is shown in SEQ ID NO: 12.

The amino acid sequence of 14G2a-CAR-7×2b is shown in SEQ ID NO: 13. The nucleotide sequence of 14G2a-CAR-7×2b is shown in SEQ ID NO: 14.

### Example 2. Construction of Vector Expressing Chimeric Antigen Receptor

(1) The CAR gene was optimized according to the theoretical protein sequence of the CAR gene, so that it can be efficiently expressed in human cells. The CAR gene was synthesized after codon optimization and by whole gene synthesis by GUANGZHOU IGE BIOTECHNOLOGY LTD.
(2) The synthesized CAR gene and the empty vector pLVX-EF1-MCS were respectively double-digested with EcoRI and BamHI in a water bath at 37° C. for 30 minutes, then subjected to DNA electrophoresis by using 1.5% agarose gel. An agarose gel kit from Tiangen Biotech (Beijing) Co., Ltd was used for purification and recovery.
(3) The vector pLVX-EF1-MCS and the CAR gene were ligated.

The ligation system is shown in Table 1.

TABLE 1

| Component | Addition amount (μl) |
|---|---|
| vector pLVX-EF1-MCS | 2 (50 ng) |
| CAR gene | 10 (150 ng) |
| T4 DNA ligation buffer | 2 |
| T4 DNA ligase (NEB) | 1 |
| dd H₂O | 5 |
| total | 20 |

The ligation reaction was conducted at 22° C. for 1 hour. The ligated product was directly transformed into Escherichia coli Stbl3 competent cells. 200 μl of the transformed products were seeded onto a LB plate containing ampicillin. The LB plate was placed upside-down in an incubator at 37° C. overnight. In the next morning, three single colonies were randomly selected for colony PCR identification The positive colonies were sent for sequencing.

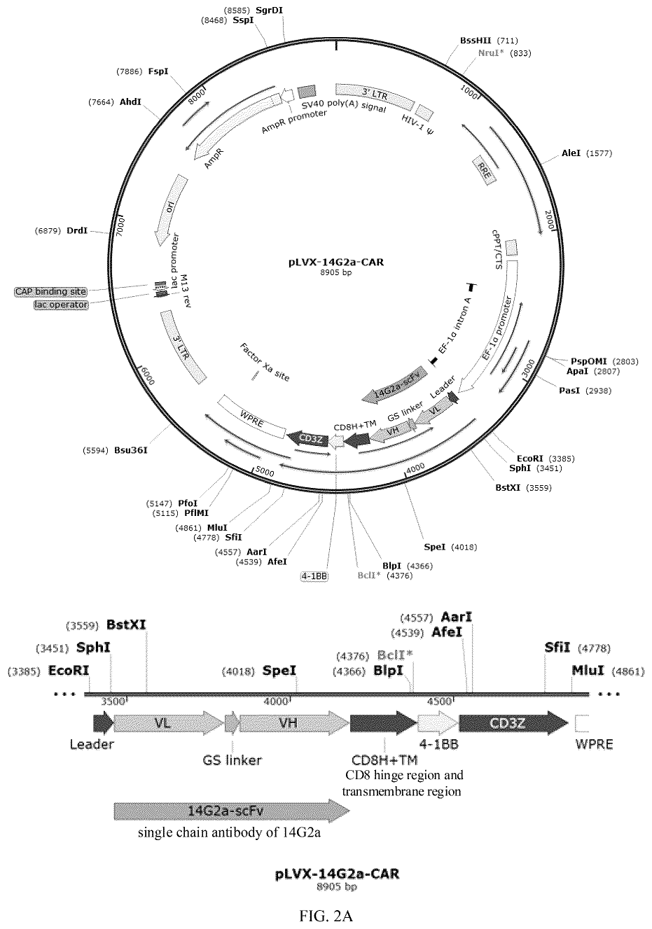

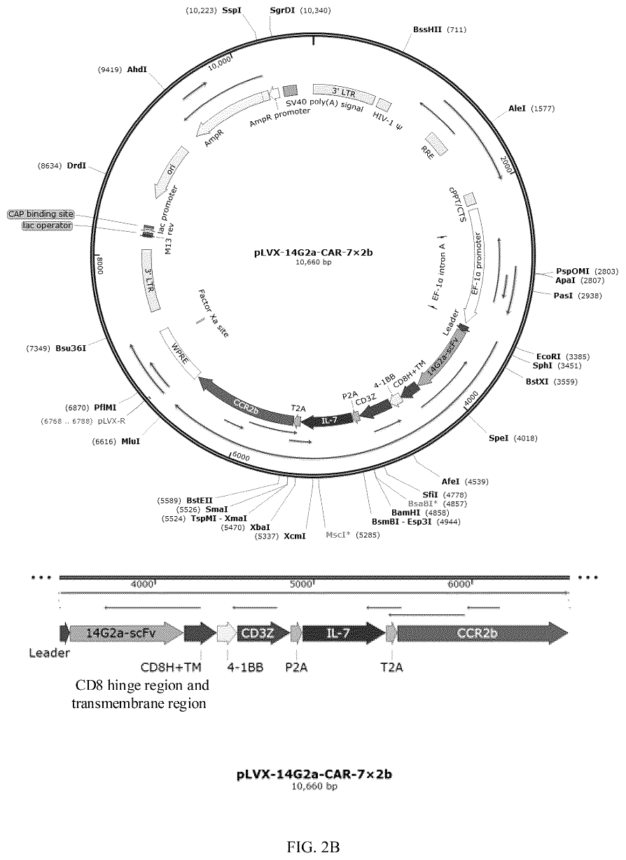

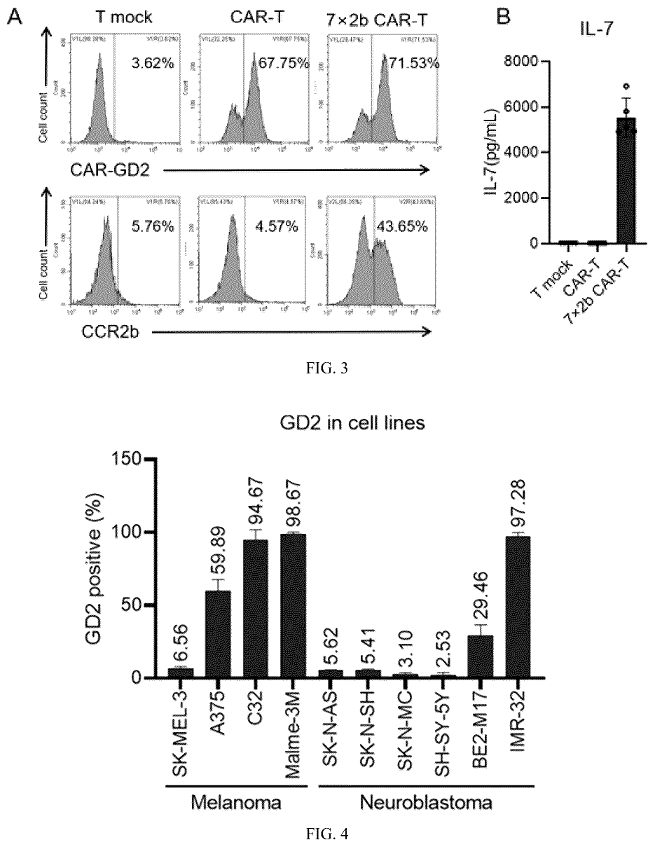

The vector map of the conventional chimeric antigen receptor lentiviral expression vector pLVX-14G2a-CAR is shown in FIG. 2A. The vector map of the chimeric antigen receptor lentiviral expression vector pLVX-14G2a-CAR-7×2b co-expressing IL-7 and CCR2b (7×2b) is shown in FIG. 2B.

### Example 3. Package of Lentivirus

The lentiviral expression vectors obtained in Example 2 were packaged respectively with a four-plasmid system according to the following steps.
(1) The four-plasmid system expresses gag/pol, Rev, VSV-G and the CAR expression vector constructed in the present disclosure which are required for packaging lentivirus respectively. The four plasmids were used for transient transfection of 293 T cells, with a DNA content of 2 μg/mL.
(2) The above plasmids were mixed with a PEI transfection reagent and the mixture was added to a certain volume of serum-free Dulbecco's modified Eagle medium (DMEM). The mixture was mixed uniformly and allowed to stand for 15 minutes, and then added to a T75 culture bottle seeded with 293T cells, gently mixed, and cultured in a cell incubator containing 5% CO₂ at 37° C. for 6 hours.
(3) After 6 hours, the culture medium was replaced with a fresh medium for continuous culture. 10 mM of butyric acid sodium solution was added. After 72 hours, the supernatant of the cultured lentivirus was collected and purified for detecting.

### Example 4. Preparation and Identification of CAR-T Cell

Peripheral blood mononuclear cells (PBMCs) were extracted from peripheral blood of a healthy human by using peripheral blood lymphocyte separation solution Ficoll. Human T cells were isolated with α-CD3/α-CD28 antibody-coated magnetic beads at a ratio of T cells to magnetic beads of 1:1, and then cultured in GT-T551 H3 Culture medium (Takara) containing 10% serum and 100 μg/mL IL-2. After 24 hours, activated human CD3+ T cells were counted. 0.1×10⁶ T cells and the virus concentrate at a multiplicity of infection (MOI) of 10 were added into each well, and then placed in a cell incubator. The culture medium was changed with a fresh culture medium after 4 hours. Then, the fresh culture medium was supplemented every 2 to 3 days. The prepared CAR-T cells are designate as conventional CART cells and 7×2b CAR T cells, respectively. The non-infected human T cells (Mock T) were used as a negative control.

After 5 days of transduction, the T cells were washed with a flow cytometry wash solution made of 50 mL PBS and 1 mL fetal bovine serum, and then centrifuged. The cell precipitates were resuspended with 100 μL of FITC-labeled protein L or anti-CCR2b, each with a working concentration of 3 μg/mL, then incubated at 4° C. for 60 minutes. After that, the cells were washed with a flow cytometry wash solution for 3 times. The expressions of CAR and CCR2b on the surface of T cells were detected by flow cytometry. The supernatant was detected for the secretion level of IL-7. According to the requirements of Cytokine ELISA detection kit (i.e., Human IL-7 ELISA KIT), the secretion level of human IL-7 cytokine by CAR-T cells was detected by the double antibody sandwich method. The chromogenic substrate was developed, and then OD values at 450 nm were obtained using a microplate reader. The corresponding concentrations of cytokine in each sample were calculated according to a standard curve.

To further verify whether the 7×2b CART cell can express CCR2b and effectively secrete IL-7, FITC-protein L (1:100) was used to detect the expression of CAR, and APC anti-human CD192 (CCR2) antibody (biolegend; 1:20) was used to detect the expression of CCR2b. It is shown that the expression of CAR detected by the protein L is 3.62% for Mock T, 67.75% for the conventional 14G2a-CAR-T, and 71.53% for the 7×2b CAR-T co-expressing IL-7 and CCR2b. In addition to CAR, the 7×2b CAR-T cell can also express CCR2b, referring to panel A of FIG. 3. The T cells were cultured in H3 medium without IL-7. The culture supernatant of T cells was taken on Day 5 for detecting the concentration of IL-7 in the supernatant by the ELISA method. It is shown that the 7×2b CAR-T cells can secrete a large amount of IL-7, but IL-7 secretion is undetectable in the control T cells without transduction of lentivirus (i.e., Mock T) or in the CAR-T cells transduced with only the conventional CAR structure, referring to panel B of FIG. 3.

### Example 5. Determination of In Vitro Anti-Tumor Activity of CAR-T Cell

It has been reported previously that the GD2 antigen can be expressed in melanoma and neuroblastoma. The expression of GD2 in the cell lines was detected by using anti-Ganglioside GD2 antibody [14.G2a] (ab68456) in this Example. As shown in FIG. 2A, in the melanoma cell lines, the GD2 antigen is highly expressed in the cell lines other than SK-MEL3 cell line; while in the neuroblastoma cell lines, the GD2 antigen is lowly expressed in the cell lines other than BE2-M17 and IMR-32 cell lines.

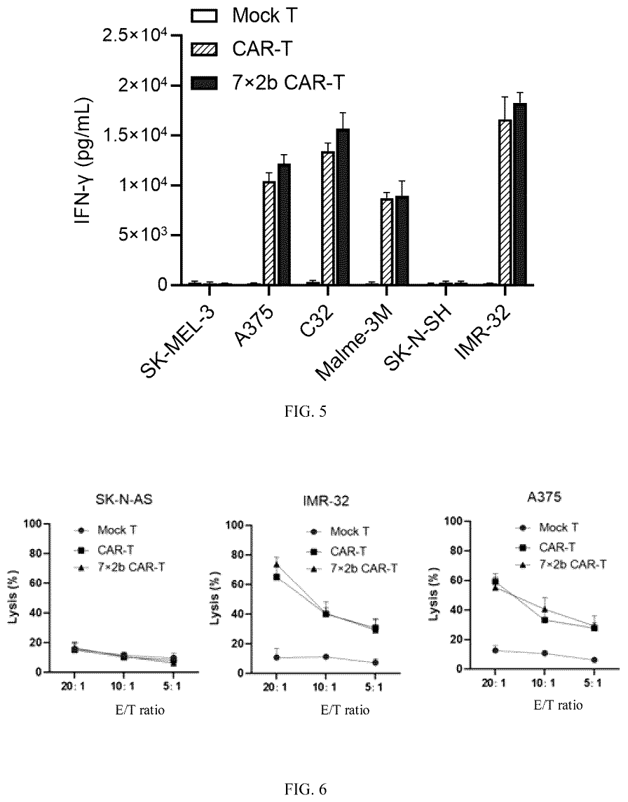

The non-transduced control T cells, classic CART cells and 7×2b CAR-T cells were co-cultured with different GD2-expressing tumor cells, respectively. The effector-target (E/T) ratio of CAR-T cells to target tumor cells is 10:1. After co-incubation in a 96-well plate for 12 hours, IFN-γ level in the supernatant was detected using an ELISA kit. In both the conventional CAR-T cell group and the 7×2b CAR-T cell group, a large amount of IFN-γ was secreted after co-incubation with the GD2-positive tumor cells, while almost no IFN-γ was secreted after co-incubation with the GD2-negative tumor cells, as shown in FIG. 5, indicating that the 7×2b CAR-T cell group and conventional CAR-T cell group can be specifically activated by the GD2-expressing tumor cells, and thus can secrete inflammatory cytokine IFN-γ, and produce immunotoxicity effects on the GD2-positive tumor cells. In addition, after co-incubation with the GD2-positive tumor cells, the secretion level of IFN-γ in the 7×2b CAR-T cell group is higher than that in the conventional CAR-T cell group, with not statistically significant difference.

The killing rates (%) to the three tumor cell lines by CAR-T cells at different effector-target ratios were determined by a luciferase assay. SK-N-AS cells, IMR-32 cells and A375 cells are previously transduced with the luciferase gene respectively to construct cell lines stably expressing luciferase. The T cells in each group were incubated with the three target tumor cells at different E/T ratios for 4 hours. It is shown that there is no significant difference in the killing effects between the 7×2b CAR-T cell group and the conventional CAR-T cell group. The CAR-T cells can kill the GD2-positive IMR-32 cells and A375 cells, but cannot kill the GD2-negative SK-N-AS cells, exhibiting a good specificity (FIG. 6).

### Example 6. Enhanced Survival and Migration Ability of CAR-T Cells by the Expression of IL-7 and CCR2b

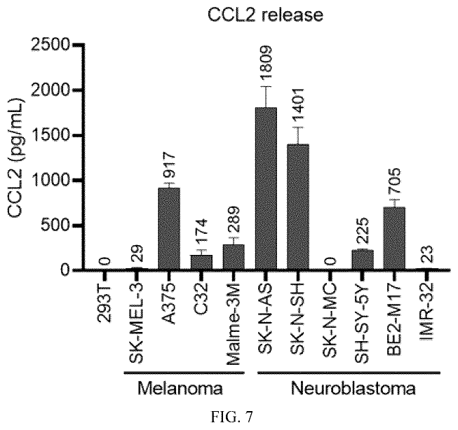

C-C motif Chemokine ligand 2 (CCL2), secreted by various tumor cells, is a member of the chemokine family that is earliest discovered and widely researched, and has a strong chemotaxis to mononuclear cells, memory T cells, and the like. Therefore, CCR2b, as a CCL2 receptor, is introduced into the structure of CAR to improve the chemotaxis of the CAR-T cells. The secretion levels of CCL2 by tumor cells were detected by the ELISA method. The results are shown in FIG. 7.

It is shown that almost no CCL2 is secreted by 293T, SK-MEL-3, SK-N-MC or IMR-32 cells; CCL2 is slightly secreted by C32, Malme-3M or SH-SY-5Y cells; and CCL2 is largely secreted by A375, SK-NS-AS, SK-N-SH or BE2M17 cells. The SK-NS-AS cells secreted the largest amount of CCL2.

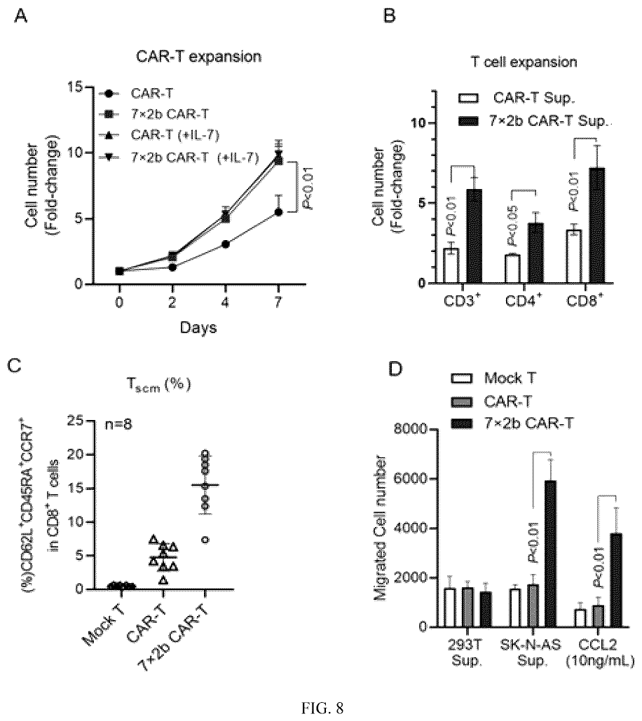

Cytokine IL-7 can effectively promote the proliferation of T cells. In order to further validate the function of the 7×2b CAR-T cells, the proliferation ability of CAR-T cells is first tested. As shown in panel A of FIG. 8, from Day 2 after antigen activation, the 7×2b CAR-T cells has proliferation ability significantly superior to that of the conventional CAR-T cells. When cultured under the addition of 10 ng/mL of IL-7, the expansion ability of the conventional CAR-T cells is improved, indicating that IL7 can enhance the expansion of the CAR-T cells. However, there was no change in cell proliferation of the 7×2b CAR-T cells when cultured under the addition of IL7, indicating that the IL7 secreted by the 7×2b CAR-T cells is sufficient to satisfy the demand for expansion.

The effect of CAR-T cell supernatant on stimulating the expansion of T cells was further analyzed. The supernatant of conventional CAR-T cells or 7×2b CAR-T cells after culture was collected and then mixed with a fresh medium (TAKARA GT-T551 H3 serum-free medium+2% autologous serum+300 U/mL IL2) at a ratio of 1:1. T cells derived from PBMC in a concentration of 1×10⁶ were added with the mixed culture medium, and the above mixed culture medium was supplemented every 2 to 3 days. After continuous culture for 7 days, the expansion ratios of CD3+, CD4+ and CD8+ subsets of T cells were detected by the flow cytometry. It is shown that T cells can be expanded by the supernatant in both the conventional CAR-T group and the 7×2b CAR-T group, and the numbers of CD3+, CD4+ and CD8+ T cells expanded in the 7×2b CAR-T group is more than those in the conventional CAR-T group, with the largest expansion number for CD8+ T cells, as shown in panel B of FIG. 8.

Since T cell subsets play an important role in the efficacy of CAR-T immunotherapy, the above three groups of T cells were cultured independently for 8 times, and then the distributions of T cell subsets were compared by the flow cytometry. It is shown that in the 7×2b CAR-T group, the proportion of T memory stem cells (Tscm) (CAR⁺CD62L⁺CD45RA⁺CCR7⁺) in CD8+ T cells is significantly higher than that in Mock T cell group and the conventional CAR-T cell group, with statistically significant difference (P<0.001), indicating that the 7×2b CAR-T cells have good persistence, as shown in panel C of FIG. 8.

The chemotactic ability of CCL2 in the supernatant of tumor cells to CAR-T cells was further evaluated. The SK-N-AS cell, which can secrete the largest amount of CCL2, was selected as the research object, and 293 T cell was used as a negative control. After the cells with a concentration of 5×10⁶ cells per well were cultured in a serum free medium for 48 hours, the supernatant was collected and added in the lower chamber of Transwell. Among them, one group with 10 ng/mL of CCL2 added in the serum-free medium was served as a positive control. 3×10⁴ of Mock T cells, conventional CAR-T cells or 7×2b CAR-T cells were respectively added in the upper chambers of Transwell and cultured for 24 hours. Then the T cells migrated into the lower chambers were counted by the flow cytometry. It is shown that the supernatant of 293 T cells can facilitate a small number of the T cells to migrate into the lower chambers, but there is no significant difference in the migrated numbers of the T cells. However, in the wells with addition of the SK-N-AS cell supernatant, the number of the 7×2b CAR-T cells migrated into the lower chamber is larger than that of Mock T cells and the conventional CAR-T cells, with statistically significant difference(P<0.01). The addition of 10 ng/mL CCL2 in the lower chamber can also cause a substantial migration of T cells, as shown in panel D of FIG. 8. It is suggested that the CCR2b receptor in the 7×2b CAR T cells can be induced by CCL2 secreted by SK-N-AS cells, which promotes the chemotaxis of the 7×2b CAR-T cells towards the supernatant containing CCL2.

### Example 7. In Vivo Anti-Tumor Activity of 7×2b CAR-T Cells

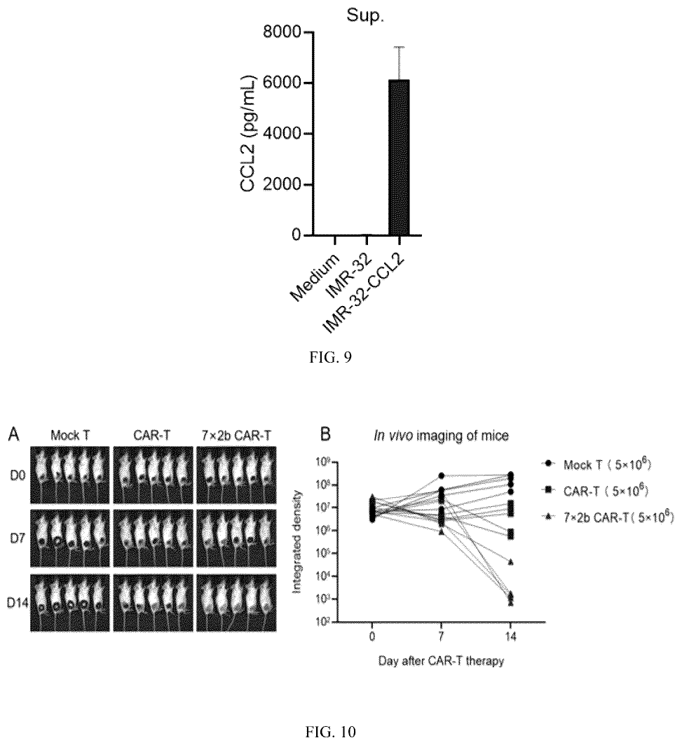

The 7×2b CAR-T cells were further studied for inhibiting tumors in vivo. IMR-32 cell, which scarcely secretes CCL2, was selected as the research object. A stably transfected cell line IMR-32-CCL2 was constructed to express CCL2 gene and a luciferase marker. As shown in FIG. 9, a high concentration of CCL2 is detected in the culture supernatant of IMR-32-CCL2 cells by the ELISA method, indicating that the stably transfected cell line constructed can secrete CCL2, and the construction is successful.

The stably transfected cell line IMR-32-CCL2 was injected subcutaneously into nude mice, with a concentration of 5×10⁶ cells per mouse, to form a tumor. After 10 days, the mice were divided into a Mock T group, a CAR-T group and a 7×2b CAR-T group, with 5 nude mice in each group. The Mock T cells, conventional CAR-T cells and 7×2b CAR-T cells were re-infused into the nude mice via the tail vein, with a concentration of 5×10⁶ cells per mouse. The nude mice were imaged in vivo on Day 0, Day 7 and Day 14. The bioluminescence imaging results are as shown in panel A of FIG. 10. Compared with the Mock T group or the conventional CAR-T group, the 7×2b CAR-T cells can effectively inhibit tumor growth, and cause regression of the tumor in mice on Day 14. The corresponding quantitative analysis of fluorescence intensity is shown in panel B of FIG. 10.

In addition, the tail venous bloods of the nude mice in each group were collected for detecting the contents of various human cytokines by the flow cytometry-based BCA method. As shown in FIG. 11, the expressions of IFN-γ, IL2 and Gzms-B are all increased on day 14 after re-infusion of the 7×2b CAR-T cells, indicating that the 7×2b CAR-T cells have the tumor-killing effect. IL-7 can be only found in the venous bloods of the mice in the 7×2b CAR-T group, indicating that IL-7 is secreted by the 7×2b CAR-T cells. CCL2 is detectable in the venous bloods of the mice in each group, with no significant difference in the level of CCL2, suggesting that CCL-2 is secreted by the inoculated IMR-32-CCL2 cells.

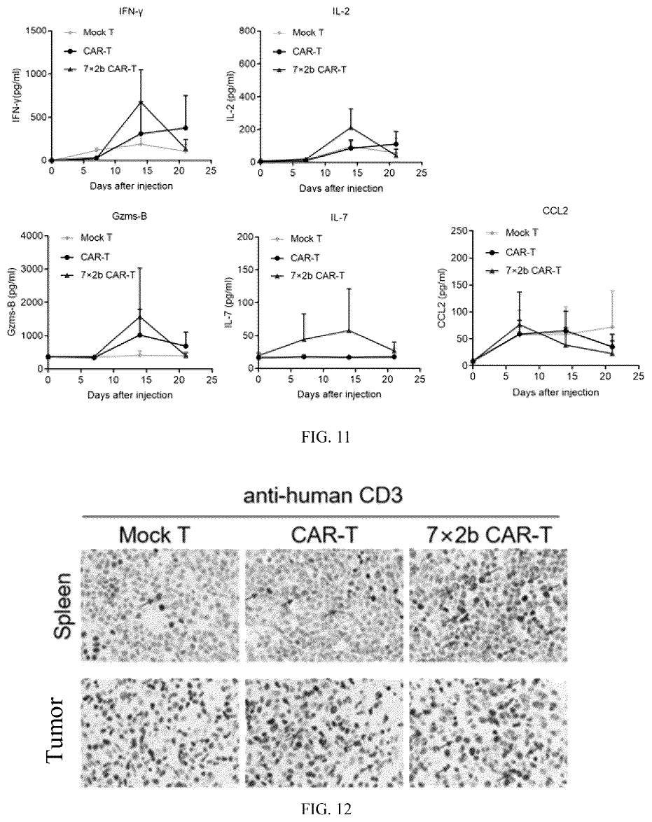

The expression of human CD3 can reflect the number of T cells re-infused in the spleen of the mice and the infiltration extent in the tumor. On day 7, the spleen and the tumor mass of the nude mice were respectively taken for immunohistochemical tests. The results show that the largest number of the 7×2b CAR-T cells exist in the spleen, as shown in FIG. 12, indicating that the 7×2b CAR-T cells have a strong expansion ability, and are more prone to migrate to a tumor site than the Mock T cells and the CAR T cells.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all of them fall within the protection scope of the present disclosure. Therefore, the protection of the present disclosure shall be defined by the appended claims.

1. A fusion protein comprising a chimeric antigen receptor, wherein the fusion protein comprises a chimeric antigen receptor, a 2A peptide, IL-7, a 2A peptide and CCR2b which are sequentially linked in series.

2. The fusion protein according to claim 1, wherein the IL-7 comprises an amino acid sequence as set forth in SEQ ID NO: 2; and the CCR2b comprises an amino acid sequence as set forth in SEQ ID NO: 3.

3. The fusion protein according to claim 1, wherein the 2A peptide is a T2A peptide comprising an amino acid sequence as set forth in SEQ ID NO: 4.

4. The fusion protein according to claim 1, wherein the chimeric antigen receptor comprises A) a se-FIT region, B) a hinge region, C) a transmembrane region, and D) an intracellular signaling region.

5. The fusion protein according to claim 4, wherein the hinge region is a hinge region of CD8α, preferably comprising an amino acid sequence as set forth in SEQ ID NO: 5.

6. The fusion protein according to claim 4, wherein the transmembrane region is any one selected from α, β or ζ chain of a T cell receptor, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB(CD137), GITR, CD40, BAFFR, HVEM(LIGHTR), SLAMF7, NKp80(KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96(Tactile), CEACAM1, CRTAM, Ly9(CD229), CD160(BY55), PSGL1, CD100(SEMA4D), SLAMF6(NTB-A, Ly108), SLAM(SLAMF1, CD150, IPO-3), BLAME(SLAMF8), SELPLG(CD162), LTBR, PAG/Cbp, NKp44, NKp30, NKp46, NKG2D, and NKG2C; and preferably the transmembrane region is a CD8α transmembrane region comprising an amino acid sequence as set forth in SEQ ID NO: 6.

7. The fusion protein according to claim 4, wherein the intracellular signaling region is any one selected from CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a CD83-specific binding ligand, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, PKCθ, FcεRIγ, ZAP70 and CD3ζ, or a combination thereof; preferably, the intracellular signaling region is 4-1BB and CD3ζ; and more preferably, the 4-1BB comprises an amino acid sequence as set forth in SEQ ID NO: 7, and the CD3ζ comprises an amino acid sequence as set forth in SEQ ID NO: 8.

8. The fusion protein according to claim 4, wherein the se-FIT region is configured to target a surface marker on a solid tumor, preferably targeting GD2, and more preferably the se-FIT region comprises an amino acid sequence as set forth in SEQ ID NO: 1.

9. (canceled)

10. (canceled)

11. A T cell expressing the fusion protein of claim 1.

12. A composition comprising the T cell of claim 11 and a pharmaceutically acceptable carrier.

13. A method for preventing and/or treating a solid tumor, comprising administering a T cell expressing the fusion protein of claim 1 or a composition comprising the T cell to a subject in need thereof.


SEQUENCE LISTING (14 sequences)

SEQ ID NO: 1 - protein, 241 aa, Artificial Sequence
  amino acid sequence of sc-Fv region
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 2 - protein, 152 aa, Artificial Sequence
  amino acid sequence of IL-7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 360 aa, Artificial Sequence
  amino acid sequence of CCR2b
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 4 - protein, 21 aa, Artificial Sequence
  amino acid sequence of T2A peptide
GSGEGRGSLLTCGDVEENPGP

SEQ ID NO: 5 - protein, 45 aa, Artificial Sequence
  amino acid sequence of CD8-alpha hinge region
TTTPAPRPPTPAPTIASQPLSLRPEACRPAAGGAVHTRGLDFACD

SEQ ID NO: 6 - protein, 24 aa, Artificial Sequence
  amino acid sequence of CD8-alpha transmembrane region
IYIWAPLAGTCGVLLLSLVITLYC

SEQ ID NO: 7 - protein, 42 aa, Artificial Sequence
  amino acid sequence of 4-1BB
KRGRKKLLYIFKQPFMRPVQTTQEEDGCSCRFPEEEEGGCEL

SEQ ID NO: 8 - protein, 112 aa, Artificial Sequence
  amino acid sequence of CD3-zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 25 aa, Artificial Sequence
  amino acid sequence of signal peptide
MFHVSFRYIFGLPPLILVLLPVASS

SEQ ID NO: 10 - protein, 21 aa, Artificial Sequence
  amino acid sequence of CD8-alpha signal peptide
MALPVTALLLPLALLLHAARP

SEQ ID NO: 11 - protein, 485 aa, Artificial Sequence
  amino acid sequence of 14G2a-CAR
MALPVTALLLPLALLLHAARPDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1458 bp, Artificial Sequence
  nucleotide sequence of 14G2a-CAR
atggcactgccagtcaccgcactgctgctgccactcgcactgctcctgcatgccgctagaccagacgtggtgatgacccagactcctctgagcctcccagtgtctctgggagatcaggcttccataagttgcagatccagccagtcactcgtccacaggaatggtaacacctacctccactggtacctgcaaaagcctggtcagtctccaaagctcctgattcacaaggtttccaatagattcagtggcgtgccagaccggttttccggctcagggagtggcaccgacttcacactcaagatcagtcgggtggaggccgaggatctcggcgtctacttttgctctcagtccacacacgttccacctctcacctttggagctggtacaaagctggagctgaaaggaggaggcggctctggcggaggtggctccggtggtggcgggagcgaggttcagctgctccagagcggacctgagctcgagaagccctctgctagcgtgatgatctcttgtaaggcttccggcagtagctttaccgggtacaacatgaattgggttaggcagaacatagggaaatctctggaatggatcggagctatcgatccctattacggtggcactagttacaaccaaaagttcaagggacgcgctactctcacagtcgataagagctccagtactgcatacatgcacctcaagtctctcacatccgaagatagcgccgtgtactattgcgtcagcgggatggagtattgggggcagggaacctccgttaccgtttcttctacaactactccagctccaagacctccaactcctgcaccaaccatcgcttctcagcctctgtctctgagacccgaggcttgcaggccagccgcaggaggagcagttcatactcgcggcctcgatttcgcatgtgatatctacatctgggctccactggcaggcacctgtggagttctgctgctgagcctggtgatcactctgtattgtaagagaggtagaaagaagctgctgtacatctttaagcagccctttatgcgccctgttcagacaacacaggaggaagatgggtgctcatgcaggttcccagaggaggaagagggagggtgcgagctgagggtcaagttttctcggagcgctgacgcacctgcctatcagcagggtcagaatcagctgtacaacgagctcaatctcggcagacgcgaggagtacgacgtgctcgataaacggcgcggtcgggaccctgaaatgggaggtaagcctcgcaggaagaacccacaggagggtctgtacaacgaactgcagaaagacaagatggctgaggcctacagcgagattggcatgaagggcgagaggaggagaggaaaaggccatgacggcctgtatcagggtctgtctactgcaactaaggatacctatgatgccctgcacatgcaggctctcccacccagataa

SEQ ID NO: 13 - protein, 1071 aa, Artificial Sequence
  amino acid sequence of 14G2a-CAR-7x2b
MALPVTALLLPLALLLHAARPDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGPGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSASRGEGRGSLLTCGDVEENPGPMLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 14 - DNA, 3216 bp, Artificial Sequence
  nucleotide sequence of 14G2a-CAR-7x2b
atggcactgccagtcaccgcactgctgctgccactcgcactgctcctgcatgccgctagaccagacgtggtgatgacccagactcctctgagcctcccagtgtctctgggagatcaggcttccataagttgcagatccagccagtcactcgtccacaggaatggtaacacctacctccactggtacctgcaaaagcctggtcagtctccaaagctcctgattcacaaggtttccaatagattcagtggcgtgccagaccggttttccggctcagggagtggcaccgacttcacactcaagatcagtcgggtggaggccgaggatctcggcgtctacttttgctctcagtccacacacgttccacctctcacctttggagctggtacaaagctggagctgaaaggaggaggcggctctggcggaggtggctccggtggtggcgggagcgaggttcagctgctccagagcggacctgagctcgagaagccctctgctagcgtgatgatctcttgtaaggcttccggcagtagctttaccgggtacaacatgaattgggttaggcagaacatagggaaatctctggaatggatcggagctatcgatccctattacggtggcactagttacaaccaaaagttcaagggacgcgctactctcacagtcgataagagctccagtactgcatacatgcacctcaagtctctcacatccgaagatagcgccgtgtactattgcgtcagcgggatggagtattgggggcagggaacctccgttaccgtttcttctacaactactccagctccaagacctccaactcctgcaccaaccatcgcttctcagcctctgtctctgagacccgaggcttgcaggccagccgcaggaggagcagttcatactcgcggcctcgatttcgcatgtgatatctacatctgggctccactggcaggcacctgtggagttctgctgctgagcctggtgatcactctgtattgtaagagaggtagaaagaagctgctgtacatctttaagcagccctttatgcgccctgttcagacaacacaggaggaagatgggtgctcatgcaggttcccagaggaggaagagggagggtgcgagctgagggtcaagttttctcggagcgctgacgcacctgcctatcagcagggtcagaatcagctgtacaacgagctcaatctcggcagacgcgaggagtacgacgtgctcgataaacggcgcggtcgggaccctgaaatgggaggtaagcctcgcaggaagaacccacaggagggtctgtacaacgaactgcagaaagacaagatggctgaggcctacagcgagattggcatgaagggcgagaggaggagaggaaaaggccatgacggcctgtatcagggtctgtctactgcaactaaggatacctatgatgccctgcacatgcaggctctcccacccagatccggatccggacctggcgctactaacttttctctcctgaagcaagctggtgatgtcgaggaaaaccctggaccaatgtttcacgtctcctttcggtatatctttggactgcctccactcattctggtcctgctgccagtggcctcttcagattgtgacattgagggtaaggatggtaaacagtacgagtctgtcctgatggttagcattgaccagctcctcgactctatgaaagagattggttctaattgcctgaataacgagttcaacttcttcaagaggcatatctgcgacgccaataaggaaggtatgtttctcttcagagctgctagaaagctgcgccagttcctgaagatgaactccacaggcgattttgacctccacctgctcaaagtctcagagggaactactattctgctgaattgtactggccaggtgaagggcagaaagccagcagcactgggtgaggctcagcccactaagtcactggaagagaacaagagcctgaaagaacaaaagaaactcaacgacctgtgctttctcaaaagactgctccaggagatcaaaacctgctggaataagattctgatgggcactaaggagcacggttcagcatctagaggagagggtagaggaagtctgctcacctgtggagatgttgaagaaaaccccgggcctatgctgtccacaagtaggagccgcttcatcagaaatacaaacgaatctggagaagaggttaccacattctttgactacgattatggcgctccatgccacaagtttgatgtcaaacagataggtgctcaactcctgccacctctgtatagcctcgttttcatcttcggcttcgtgggaaatatgctcgtggtgctgattctgattaactgtaagaaactgaaatgcctgaccgacatctacctcctcaacctcgccattagcgacctgctgtttctgattaccctccctctgtgggcacatagtgctgcaaatgaatgggtgttcggtaacgctatgtgcaaactctttacagggctctatcacattgggtactttggcggtatcttcttcatcatcctgctcaccatagaccggtatctggctatcgttcatgccgtgttcgccctgaaggctaggacagtgacctttggagttgtgacaagtgttatcacttggctggtcgcagtgttcgccagtgtgccagggatcatctttaccaaatgccagaaggaagattcagtgtacgtctgcggaccatacttccctagaggttggaataatttccacaccataatgcgcaacatcctgggactcgtgctgccactgctgatcatggtgatttgttactcaggcatcctgaaaaccctgctccgctgccggaacgagaagaaacggcatagggcagtgagggttatattcacaatcatgatcgtgtactttctgttctggacaccttacaacattgtgatactcctgaacactttccaagagttcttcggtctgtctaactgtgaaagcaccagccaactcgaccaagcaacccaggttactgagacactgggcatgacacactgttgcatcaatcctatcatatacgcatttgttggcgagaaatttcggagatacctctcagtgttcttcagaaaacacataactaagaggttctgtaaacagtgccctgtcttctatcgcgagacagtggatggagtgacaagcaccaatactccttctaccggagaacaggaggtgtccgcaggactgtaa